(19) United States
(12) Patent Application Publication
(10) Pub. No.: US 2003/0135055 A1
(43) Pub. Date: Jul. 17, 2003

(54) Aminosulfonylbiphenyl derivatives

(76) Inventors: Dieter Dorsch, Ober-Ramstadt (DE); Horst Juraszyk, Seeheim-Jugenheim (DE); Werner Mederski, Erzhausen (DE); Christos Tsaklakidis, Weinheim (DE); Sabine Bernotat-Danielowski, Bad Nauheim (DE); Guido Melzer, Hofheim Ts (DE); Johannes Gleitz, Darmstadt (DE); Christopher Barnes, Bad Soden (DE); James Vickers, Reading (GB)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/204,455

(22) PCT Filed: Feb. 22, 2001
(86) PCT No.: PCT/EP01/02034

(30) Foreign Application Priority Data
Feb. 23, 2000 (DE) 10008329.3

(51) Int. Cl. C07D 271/12; C07D 271/06; C07C 311/30

(52) U.S. Cl. 548/131; 548/132; 560/12; 562/430; 558/413

(57) ABSTRACT

The invention relates to compounds of the formula I

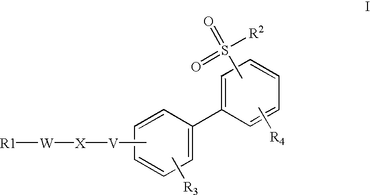

in which R¹, R², R³, R⁴, W, X and V have the meaning indicated in the text. The compounds act as inhibitors of factors Xa and VIIa and can therefore be employed for the control and prevention of thromboembolic disorders such as thrombosis, myocardial infarct, arteriosclerosis, inflammation, apoplexy, angina pectoris, restenosis after angioplasty and intermittent claudication.

The invention relates to compounds of the formula I

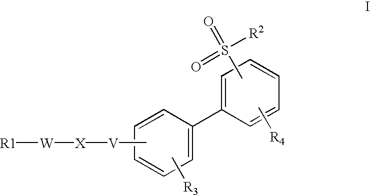

in which:

R¹ is phenyl or naphthyl, which is substituted by —C(═NH)NH₂ (which can also be monosubstituted by —COA, —CO—[C(R⁶)₂—Ar′, —COOA, —OH or by a conventional amino protective group), —NHC(═NH)—NH₂,

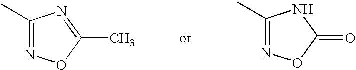

and which can optionally be substituted by —A, —OR⁵, —N(R⁵)₂, —NO₂, —CN, —Hal, —NR⁵COA, —NR⁵COAr′, —NR⁵SO₂A, —NR⁵SO₂Ar′, —COOR⁵, —CON(R⁵)₂, CONR⁵Ar′, COR⁶, —COAr′ or S(O)ₙA;

R² is —N(R⁵)₂, —NR⁵COA, —NR⁵COAr, —NR⁵COOR⁵;

R³,R⁴ independently of one another are —H, —A, —OR⁵, —N(R⁵)₂, —NO₂, —CN, —Hal, —NR⁵COA, —NR⁵COAr′, —NR⁵SO₂A, —NR⁵SO₂Ar′, —COOR⁵, —CON(R⁵)₂, —CONR⁵Ar′, —COR⁶, —COAr′, —S(O)Ar′, S(O)ₙA;

R⁵ is —H, —A, —C(R⁶R⁷)Ar′ or —C(R⁶R⁷)Het;

R⁶,R⁷ independently of one another are —H, —A or —(CH₂)ₗ—Ar′;

R⁸ is H or A;

X is —O—, —NR⁵—, —CONR⁵—, —N(SO₂Ar)—, —N(SO₂Het)—;

W is —(CR⁶R⁷)ₙ,—(OCR⁶R⁷)—, 1,3-phenylene, 1,3-phenylene-C(R⁶)₂-, 1,4-phenylene, 1,4-phenylene-C(R⁶)₂-;

V is —(C(R⁶)₂)m⁻;

A is alkyl having 1 to 20 C atoms, in which one or two CH₂ groups can be replaced by O or S atoms or by —CH═CH— groups and also 1 to 7H atoms can be replaced by F;

Ar is phenyl or naphthyl, which is unsubstituted or mono-, di- or trisubstituted by —A, —Ar′, —Het,—-OR⁵, —N(R⁵)₂, —NO₂, —CN, —Hal, —NR⁵COA, —NR⁵COAr, —NR⁵SO₂A, —NR⁵SO₂Ar′, —COOR⁵, —CON(R⁵)₂, —CONR⁵Ar′, —COR⁶, —COAr′ or —S(O)ₙA;

Ar′ is phenyl or naphthyl, which is unsubstituted or mono-, di- or trisubstituted by —A, —OR⁸, —N(R⁸)₂, —NO₂, —CN, —Hal, —NR⁸COA, —NR⁶SO₂A, —COOR⁸, —CON(R⁸)₂, —COR⁸, —SO₂NR⁸ or —S(O)ₙA;

Het is a mono- or binuclear saturated, unsaturated or aromatic heterocycle having 1 to 4 N, O and/or S atoms, bonded via N or C, which can be unsubstituted or mono-, di or trisubstituted by —A, —OR⁶, —N(R⁶)₂, —NO₂, —CN, —Hal, —NR⁶COA, —NR⁶SO₂A, —COOR⁶, —CON(R⁶)₂, —COR⁶, —SO₂NR⁶, —S(O)ₙA and/or carbonyl oxygen;

Hal is —F, —Cl, —Br or —l;

l is 0, 1, 2, 3, 4, or 5;

m is 0 or 1;

n is 0, 1 or 2;

o is 1 or 2;

and their pharmaceutically tolerable salts and solvates.

The invention also relates to the optically active forms, the racemates, the diastereomers and the hydrates and solvates, e.g. alcoholates, of these compounds.

The invention was based on the object of finding novel compounds having valuable properties, in particular those which can be used for the production of medicaments.

It has been found that the compounds of the formula I and their salts have very valuable pharmacological properties together with good tolerability. In particular, they exhibit factor Xa-inhibiting properties and can therefore be employed for the control and prevention of thromboembolic disorders such as thrombosis, myocardial infarct, arteriosclerosis, inflammation, apoplexy, angina pectoris, restenosis after angioplasty and intermittant claudication.

The compounds of the formula I according to the invention can furthermore be inhibitors of the clotting factors factor VIIa, factor IXa and thrombin of the blood-clotting cascade.

Compounds which act as inhibitors on factor Xa are described, for example, in EP 540 051, WO 96/10022, WO 97/08165, WO 96/40679 and WO 98/28282.

The antithrombotic and anticoagulent effect of the compounds according to the invention is to be attributed to the inhibitory action against the activated crossing protease, known under the name factor Xa, or to the inhibition of other activated serine proteases such as factor VIIa, factor IXa or thrombin.

Factor Xa is one of the proteases which is involved in the complex process of blood clotting. Factor Xa catalyses the conversion of prothrombin into thrombin. Thrombin cleaves fibrinogen into fibrin monomers, which after crosslinking contribute in elementary form to thrombus formation. Activation of thrombin can lead to the occurrence of thromboembolic diseases. Inhibition of thrombin, however, can inhibit the fibrin formation involved in the thrombus formation.

The inhibition of thrombin can be measured, for example, by the method of G. F. Cousins et al. in Circulation 1996, 94, 1705-1712.

Inhibition of factor Xa can thus prevent thrombin being formed.

The compounds of the formula I according to the invention and their salts intervene in the blood-clotting process by inhibition of factor Xa and thus inhibit the formation of thrombi.

The inhibition of factor Xa by the compounds according to the invention and the anticoagulant and antithrombotic activity can be determined by customary in vitro or in vivo methods. A suitable procedure is described, for example, by J. Hauptmann et al. in Thrombosis and Haemostasis 1990, 63, 220-223.

The measurement of the inhibition of factor Xa can be carried out, for example, by the method of T. Hara et al. in Thromb. Haemostas. 1994, 71, 314-319. After binding to tissue factor, the clotting factor VIIa initiates the extrinsic part of the clotting cascade and contributes to the activation of factor X to factor Xa. Inhibition of factor Vila thus prevents the formation of factor Xa and thus subsequent thrombin formation.

The inhibition of factor VIIa by the compounds according to the invention and the anticoagulant and antithrombotic activity can be determined by customary in vitro or in vivo methods. A customary procedure for measurement of the inhibition of factor VIIa is described, for example, by H. F. Ronning et al. in Thrombosis Research 1996, 84, 73-81.

The clotting factor IXa is generated in the intrinsic clotting cascade and is likewise involved in the activation of factor X to factor Xa. Inhibition of factor IXa can therefore prevent factor Xa being formed in a different manner.

The inhibition of factor IXa by the compounds according to the invention and the anticoagulant and antithrombotic activity can be determined by customary in vitro or in vivo methods. A suitable procedure is described, for example, by J. Chang et al. in Journal of Biological Chemistry 1998, 273, 12089-12094.

The compounds of the formula I can be employed as pharmaceutical active compounds in human and veterinary medicine, in particular for the control and prevention of thromboembolic disorders such as thrombosis, myocardial infarct, arteriosclerosis, inflammation, apoplexy, angino pectoris, restenosis after angioplasty and intermittent claudication.

Particularly active inhibitors of factor Xa or factor VIIa have turned out to be compounds of the formula II:

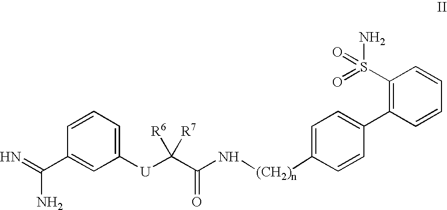

in which in addition:

U is —O— or —CH₂—.

The following compounds are of particularly great importance:

2-(3-Carbamimidoylphenoxy)acetic acid (2′-sulfamoylbiphenyl-4-yl)amide (1),

2-(3-Carbamimidoylphenoxy)-2-phenyl-N-(2′-sulfamoylbiphenyl-4-yl)acetamide (2),

2-(3-Carbamimidoylphenoxy)valeric acid (2′-sulfamoylbiphenyl-4-yl)amide (3),

2-(3-Carbamimidoylphenoxy)hexanoic acid (2′-sulfamoylbiphenyl-4-yl)amide (4),

2-(3-Carbamimidoylphenoxy)heptanoic acid (2′-sulfamoylbiphenyl-4-yl)amide (5),

2-(3-Carbamimidoylphenoxy)-3-methyl-N-(2′-sulfamoylbiphenyl-4-yl)butyramide (6);

2-(3-Carbamimidoylphenoxy)-2-methylvaleric acid (2′-sulfamoylbiphenyl-4-yl)amide (7),

2-(3-Carbamimidoylphenoxy)-2-phenyl-N-(2′-sulfamoylbiphenyl-4-yl)acetamide (8),

2-(3-Carbamimidoylphenoxy)-4-phenyl-N-(2′-sulfamoylbiphenyl-4-yl)butyramide (9),

2-(3-Carbamimidoylphenoxy)-2-methyl-N-(2′-sulfamoylbiphenyl-4-yl)propionamide (10),

3-(3-Carbamimidoylphenoxy)propionic acid (2′-sulfamoylbiphenyl-4-yl)amide (11),

2-(3-Carbamimidoylbenzyl) pentanoic acid (2′-sulfamoylbiphenyl-4-yl)amide (12),

3-(3-Carbamimidoylphenyl)-2-phenyl-N-(2′-sulfamoylbiphenyl-4-yl)propionamide (13),

2-Benzyl-3-(3-carbamimidoylphenyl)-N-(2′-sulfamoylbiphenyl-4-yl)propionamide (14),

2-(3-Carbamimidoylbenzyl)-N-(2′-sulfamoylbiphenyl-4-yl)butyramide (65), 2-(3-Carbamimidoylbenzyl)-4-methylpenanoic acid (2′-sulfamoylbiphenyl-4-yl)amide (66),

2-(3-Carbamimidoylphenoxy)acetic acid (2′-sulfamoyibiphenyl-4-ylmethyl)amide (15),

2-(3-Carbamimidoylphenoxy)-N-(2′-sulfamoylbiphenyl-4-ylmethyl)propionamide (16),

2-(3-Carbamimidoylphenoxy)-N-(2′-sulfamoylbiphenyl-4-ylmethyl)butyramide (17),

2-(3-Carbamimidoylphenoxy) pentanoic acid (2′-sulfamoylbiphenyl-4-ylmethyl)amide (18),

2-(3-Carbamimidoylphenoxy)-3-methyl-N-(2′-sulfamoylbiphenyl-4-ylmethyl)butyramide (19),

2-(3-Carbamimidoylphenoxy)-4-methylpentanoic acid (2′-sulfamoyl-biphenyl-4-ylmethyl)amide (20),

2-(3-Carbamimidoylphenoxy)-2-phenyl-N-(2′-sulfamoylbiphenyl-4-ylmethyl)acetamide (21),

2-(3-Carbamimidoylphenoxy) propionic acid (3′-sulfamoylbiphenyl-4-yl)amide (22),

2-(3-Carbamimidoylphenoxy)butyric acid (3′-sulfamoylbiphenyl-4-yl)amide (23),

2-(3-Carbamimidoylphenoxy)valeric acid (3′-sulfamoylbiphenyl-4-yl)amide (24),

2-(3-Carbamimidoylphenoxy)-4-methylvaleric acid (3′-sulfamoylbiphenyl-4-yl)amide (25),

2-(3-Carbamimidoylphenoxy)-2-phenylacetic acid (3′-sulfamoylbiphenyl-4-yl)amide (26),

2-(3-Carbamimidoylphenoxy)-N-(3′-sulfamoylbiphenyl-3-yl)butyramide (27),

2-(3-Carbamimidoylphenoxy)pentanoic acid (3′-sulfamoylbiphenyl-3-yl)amide (28),

2-(3-Carbamimidoylphenoxy)-4-methylpentanoic acid (3′-sulfamoyl-biphenyl-3-yl)amide (29),

2-(3-Carbamimidoylphenoxy)-2-phenyl-N-(3′-sulfamoylbiphenyl-3-yl)acetamide (30),

2-(4-Carbamimidoylphenoxy)pentanoic acid (2′-sulfamoylbiphenyl-4-yl)amide (31),

2-(4-Carbamimidoylphenoxy)-2-phenyl-N-(2′-sulfamoylbiphenyl-4-yl)acetamide (32),

3-Carbamimidoylbenzoic acid (2′-sulfamoylbiphenyl-4-yl)amide (33),

2-(3-Carbamimidoylphenyl)acetic acid (2′-sulfamoylbiphenyl-4-yl)amide (34),

4-Carbamimidoylbenzoic acid (2′-sulfamoylbiphenyl-4-yl)amide (35),

2-(4-Carbamimidoylphenyl)acetic acid (2′-sulfamoylbiphenyl-4-yl)amide (36),

3-(4-Carbamimidoylphenyl) propionic acid (2′-sulfamoylbiphenyl-4-yl)amide (37),

2-(4-Carbamimidoylphenoxy)acetic acid (2′-sulfamoylbiphenyl-4-yl)amide (38),

3-(3-Carbamimidoylphenyl)propionic acid (2′-sulfamoylbiphenyl-4-ylmethyl)amide (39),

2-(3-Carbamimidoylphenyl)acetic acid (2′-sulfamoylbiphenyl-4-ylmethyl)amide (40),

2-(4-Carbamimidoylphenyl)acetic acid (3′-sulfamoylbiphenyl-4-yl)amide (41),

2-(3-Carbamimidoylphenyl)acetic acid (3′-sulfamoylbiphenyl-4-yl)amide (42),

3-(3-Carbamimidoylphenyl)propionic acid (3′-sulfamoylbiphenyl-4-yl)amide (43),

2-(3-Carbamimidoylphenoxy)acetic acid (3′-sulfamoylbiphenyl-4-yl)amide (44),

4-(2′-Sulfamoylbiphenyl-3-yloxymethyl)benzamidine (45),

3-(2′-Sulfamoylbiphenyl-3-yloxymethyl)benzamidine (46),

4-(2′-Sulfamoylbiphenyl-4-yloxymethoxy) benzamidine (47),

3-(2′-Sulfamoylbiphenyl-4-yloxymethoxy)benzamidine (48),

3-(3-Carbamimidoylphenoxy)-N-(2′-sulfamoylbiphenyl-4-yl) propionamide (67),

2-(4-Carbamimidoylphenyl)acetic acid (2′-sulfamoylbiphenyl-3-yl)amide (49),

2-(3-Carbamimidoylphenyl)acetic acid (2′-sulfamoylbiphenyl-3-yl)amide (50),

3-(4-Carbamimidoylphenyl)propionic acid (2′-sulfamoylbiphenyl-3-yl)amide (51),

3-(3-Carbamimidoylphenyl)propionic acid (2′-sulfamoylbiphenyl-3-yl)amide (52),

2-(4-Carbamimidoylphenoxy)acetic acid (2′-sulfamoylbiphenyl-3-yl)amide (53),

2-(3-Carbamimidoylphenoxy)acetic acid (2′-sulfamoylbiphenyl-3-yl)amide (54),

7-(2′-Sulfamoylbiphenyl-4-yloxymethyl)naphthalene-2-carboxamidine (55),

7-(2′-Sulfamoylbiphenyl-4-yloxymethoxy)naphthalene-2-carboxamidine (56),

7-(2′-Sulfamoylbiphenyl-4-ylaminomethyl)naphthalene-2-carboxamidine (57),

7-(2′-Sulfamoylbiphenyl-3-yloxymethyl)naphthalene-2-carboxamidine (58),

3′-(2′-Sulfamoylbiphenyl-4-ylaminomethyl)biphenyl-3-carboxamidine (59),

3′-(2′-Sulfamoylbiphenyl-4-yloxymethyl) biphenyl-3-carboxamidine (60),

N-(4-Ethylbenzenesulfonyl)-3′-(2′-sulfamoylbiphenyl-4-ylaminomethyl)biphenyl-3-carboxamidine (61),

3′-(2′-Sulfamoylbiphenyl-3-yloxymethyl)biphenyl-3-carboxamidine (62),

3′-Carbamimidoylbiphenyl-3-carboxylic acid (2′-sulfamoylbiphenyl-3-yl)amide (63),

3′-Carbamimidoylbiphenyl-3-carboxylic acid (2′-sulfamoylbiphenyl-4-yl)amide (64),

2-(3-Carbamimidoylbenzyl)hexanoic acid (2′-sulfamoylbiphenyl-4-yl)amide (68),

3-{1-[(2′-Sulfamoylbiphenyl-4-ylamino)methyl]butoxy} benzamidine (69).

The molecular ion peaks of these compounds determined by FAB (Fast Atom Bombardement) mass spectroscopy are listed in the following tables. The compounds are in each case shown as trifluoroacetates.

In some cases the molecular peaks determined by ESI (Electron Spray Ionization) mass spectroscopy are also indicated. These values are marked by *.

TABLE 1
measured molecular ion peaks of synthesized active compounds

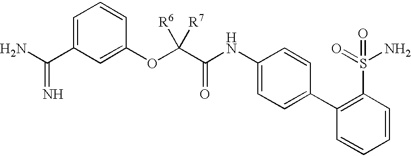

| No. | R₆ | R₇ | FAB |
|---|---|---|---|
| 1 | H | H | 425 |
| 2 | 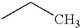 | H | 453 |
| 3 | 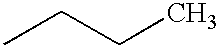 | H | 467 |
| 4 | 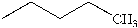 | H | 481 |
| 5 | 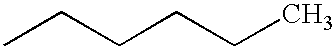 | H | 495 |
| 6 | 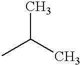 | H | 467 |
| 7 | 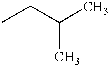 | H | 481 |
| 8 | 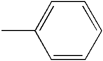 | H | *501 |
| 9 | 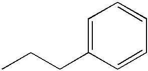 | H | 529 |
| 10 | —CH₃ | —CH₃ | 453 |

TABLE 2
measured molecular ion peaks of synthesized active compounds

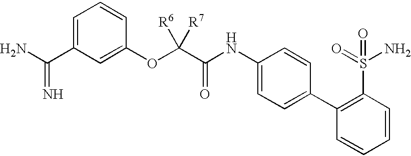

| No. | R₆ | R₇ | FAB |
|---|---|---|---|
| 11 | H | H | *423 |
| 12 | 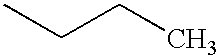 | H | *465 |
| 13 | 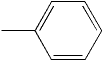 | H | *499 |
| 14 | 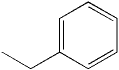 | H | *513 |
| 65 | 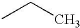 | H | *451 |
| 66 | 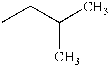 | H | *479 |

TABLE 3
measured molecular ion peaks of synthesized active compounds

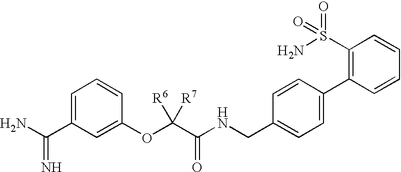

| No. | R₆ | R₇ | FAB |
|---|---|---|---|
| 15 | H | H | 439 |
| 16 | —CH₃ | H | 453 |
| 17 | 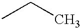 | H | 467 |
| 18 | 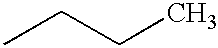 | H | 481 |
| 19 | 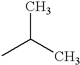 | H | 481 |
| 20 | 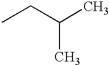 | H | 495 |
| 21 | 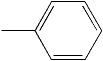 | H | 515 |

TABLE 4
measured molecular ion peaks ot synthesized active compounds

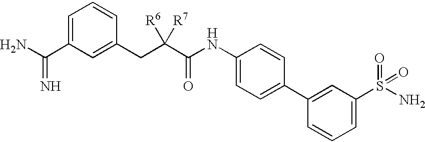

| No. | R₆ | R₇ | FAB |
|---|---|---|---|
| 22 | —CH₃ | H | 439 |
| 23 | 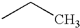 | H | 453 |
| 24 | 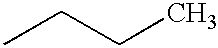 | H | 467 |
| 25 | 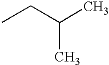 | H | 481 |
| 26 | 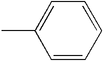 | H | 501 |

TABLE 5
measured molecular ion peaks of synthesized active compounds

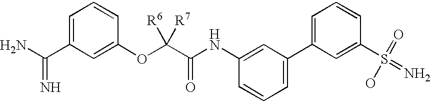

| No. | R₆ | R₇ | FAB |
|---|---|---|---|
| 27 | 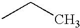 | H | 453 |
| 28 | 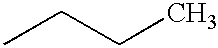 | H | 467 |
| 29 | 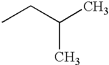 | H | 481 |
| 30 | 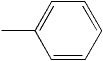 | H | 501 |

TABLE 6
measured molecular ion peaks of synthesized active compounds

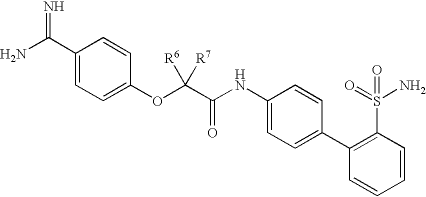

| No. | R₆ | R₇ | FAB |
|---|---|---|---|
| 31 | 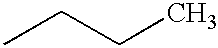 | H | — |
| 32 | 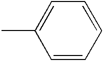 | H | 501 |

TABLE 7
measured molecular ion peaks of synthesized active compounds

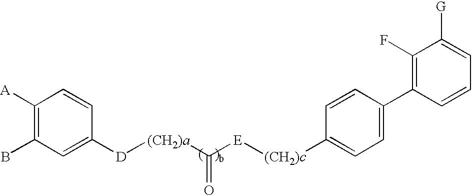

| No. | A | B | D* | E | F | G | a | b | c | FAB |
|---|---|---|---|---|---|---|---|---|---|---|
| 33 | H | 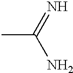 | — | —NH— | —SO₂NH₂ | —H | 0 | 1 | 0 | 395 |
| 34 | H | 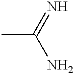 | — | —NH— | —SO₂NH₂ | —H | 1 | 1 | 0 | 409 |
| 35 | 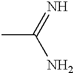 | —H | — | —NH— | —SO₂NH₂ | —H | 0 | 1 | 0 | 395 |
| 36 | 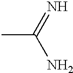 | —H | — | —NH— | —SO₂NH₂ | —H | 1 | 1 | 0 | 409 |
| 37 | 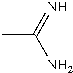 | —H | — | —NH— | —SO₂NH₂ | —H | 2 | 1 | 0 | 423 |
| 38 | 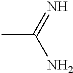 | —H | —O— | —NH— | —SO₂NH₂ | —H | 1 | 1 | 0 | 425 |
| 39 | H | 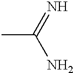 | — | —NH— | —SO₂NH₂ | —H | 2 | 1 | 1 | 437 |
| 40 | H | 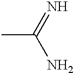 | — | —NH— | —SO₂NH₂ | —H | 1 | 1 | 1 | 423 |
| 41 | 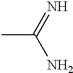 | —H | — | —NH— | —H | —SO₂NH₂ | 1 | 1 | 0 | 409 |
| 42 | H | 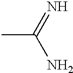 | — | —NH— | —H | —SO₂NH₂ | 1 | 1 | 0 | 409 |
| 43 | H | 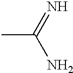 | — | —NH— | —H | —SO₂NH₂ | 2 | 1 | 0 | 423 |
| 44 | H | 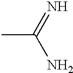 | —O— | —NH— | —H | —SO₂NH₂ | 1 | 1 | 0 | 425 |
| 45 | 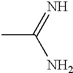 | —H | — | —O— | —SO₂NH₂ | —H | 1 | 0 | 0 | 382 |
| 46 | —H | 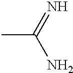 | — | —O— | —SO₂NH₂ | —H | 1 | 0 | 0 | 382 |
| 47 | 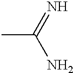 | —H | — | —O— | —SO₂NH₂ | —H | 0 | 0 | 1 | 382 |
| 48 | —H | 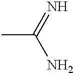 | — | —O— | —SO₂NH₂ | —H | 0 | 0 | 1 | 382 |
| 67 | —H | 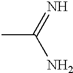 | —O— | —NH— | —SO₂NH₂ | —H | 2 | 1 | 0 | *439 |

D* - ≅ single bond

TABLE 8
measured molecular ion peaks of synthesized active compounds

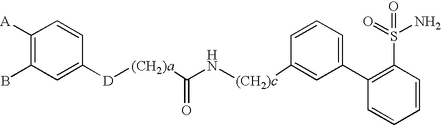

| No. | A | B | D* | a | c | FAB |
|---|---|---|---|---|---|---|
| 49 | 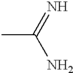 | —H | — | 1 | 0 | 409 |
| 50 | —H | 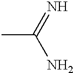 | — | 1 | 0 | 409 |
| 51 | 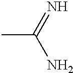 | —H | — | 2 | 0 | 423 |
| 52 | —H | 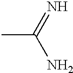 | — | 2 | 0 | 423 |
| 53 | 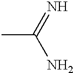 | —H | —O— | 1 | 0 | 425 |
| 54 | —H | 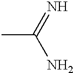 | —O— | 1 | 0 | 425 |

TABLE 9
measured molecular ion peaks of synthesized active compounds

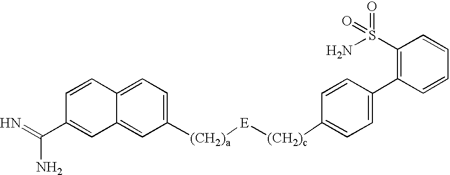

| No. | E | a | c | FAB |
|---|---|---|---|---|
| 55 | —O— | 1 | 0 | 432 |
| 56 | —O— | 0 | 1 | 432 |
| 57 | —NH— | 1 | 0 | 431 |

TABLE 10
measured molecular ion peaks of synthesized active compounds

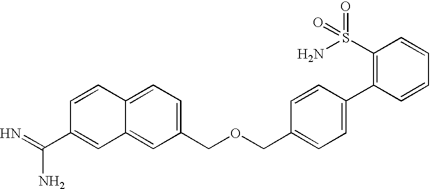

| No. | FAB |
|---|---|
| 58 | 432 |

TABLE 11
measured molecular ion peaks of synthesized active compounds

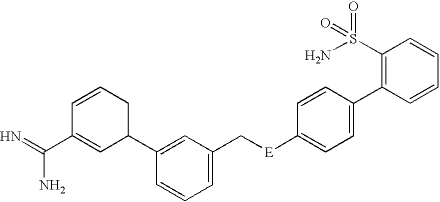

| No. | E | FAB |
|---|---|---|
| 59 | —NH— | 457 |
| 60 | —O— | 458 |
| 61 | 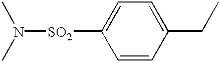 | 625 |

TABLE 12
measured molecular ion peaks of synthesized active compounds

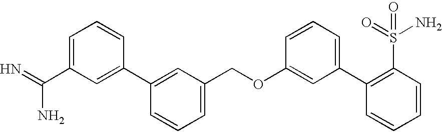

| No. | FAB |
|---|---|
| 62 | 458 |

TABLE 13
measured molecular ion peaks of synthesized active compounds

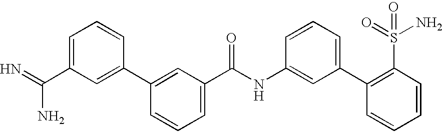

| No. | FAB |
|---|---|
| 63 | 471 |

TABLE 14
measured molecular ion peaks of synthesized active compounds

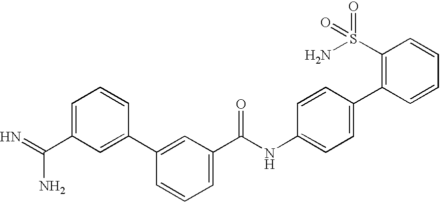

| No. | FAB |
|---|---|
| 64 | 471 |

TABLE 15
measured molecular ion peaks of synthesized active compounds

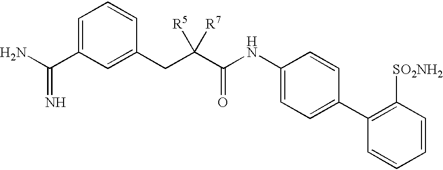

| No. | R⁶ | R⁷ | *ESI |
|---|---|---|---|
| 68 | 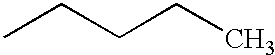 | —H | *479 |

TABLE 16
measured molecular ion peaks of synthesized active compounds

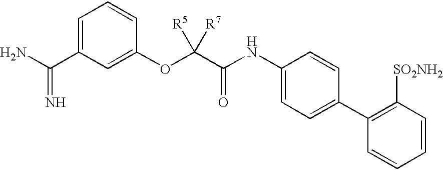

| No. | R⁶ | R⁷ | *ESI |
|---|---|---|---|
| 69 | 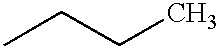 | —H | *453 |

The invention further relates to the use of the compounds of the formula I and/or their physiologically acceptable salts for the production of pharmaceutical preparations, in particular in a non-chemical way. They can be brought into a suitable dose form here together with at least one solid, liquid and/or semi-liquid vehicle or excipient and, if appropriate, in combination with one or more further active compounds.

The invention further relates to pharmaceutical preparations, comprising at least one compound of the formula I and/or one of its physiologically acceptable salts.

These preparations can be used as medicaments in human or veterinary medicine. Suitable vehicles are organic or inorganic substances which are suitable for enteral (e.g. oral), or parenteral administration or topical application and do not react with the novel compounds, for example water, vegetable oils, benzyl alcohols, alkylene glycols, polyethylene glycols, glyceryl triacetate, gelatin, carbohydrates such as lactose or starch, magnesium stearate, talc, petroleum jelly. In particular, tablets, pills, coated tablets, capsules, powders, granules, syrups, juices or drops are used for oral administration, suppositories are used for rectal administration, solutions, preferably oily or aqueous solutions, and in addition suspensions, emulsions or implants, are used for parenteral administration, and ointments, creams or powders are used for topical application. The novel compounds can also lyophilized and the lyophilizates obtained used, for example, for the production of injection preparations. The preparations indicated can be sterilized and/or can contain excipients such as lubricants, preservatives, stabilizers and/or wetting agents, emulsifiers, salts for influencing the osmotic pressure, buffer substances, colorants, flavourings and/or one or more further active compounds, e.g. one or more vitamins.

The compounds of the formula I and their physiologically acceptable salts can be used in the control and prevention of thromboembolic disorders such as thrombosis, myocardial infarct, arteriosclerosis, inflammation, apoplexy, angina pectoris, restenosis after angioplasty and intermittent claudication.

As a rule, the substances according to the invention are preferably administered here in doses of between approximately 1 and 500 mg, in particular between 5 and 100 mg, per dose unit. The daily dose is preferably between approximately 0.02 and 10 mg/kg of bodyweight. The specific does for each patient depends, however, on all sorts of factors, for example on the efficacy of the specific compound employed, on the age, bodyweight, general state of health, sex, on the diet, on the time and route of administration, on the excretion rate, pharmaceutical combination and severity of the particular disorder to which the therapy applies. Oral administration is preferred.

The compounds of the formula I and also the starting substances for their preparation are prepared by methods known per se, such as are described in the literature (e.g. in the standard works such as Houben-Weyl, Methoden der organischen Chemie [Methods of Organic Chemistry], Georg-Thieme-Verlag, Stuttgart), namely under reaction conditions which are known and suitable for the reactions mentioned. In this case, use can also be made of variants which are known per se, but not mentioned here in greater detail.

The starting substances can, if desired, also be formed in situ, such that they are not isolated from the reaction mixture, but immediately reacted further to give the compounds of the formula I. Below, a synthesis is generally presented with which compounds of the formula I can be prepared. For the preparation of specific compounds, the synthesis can be varied by the choice of suitable starting compounds. The synthesis is only intended to show by way of example a possible route for the preparation of compounds of the formula I. However, other synthesis routes can also be used for preparation.

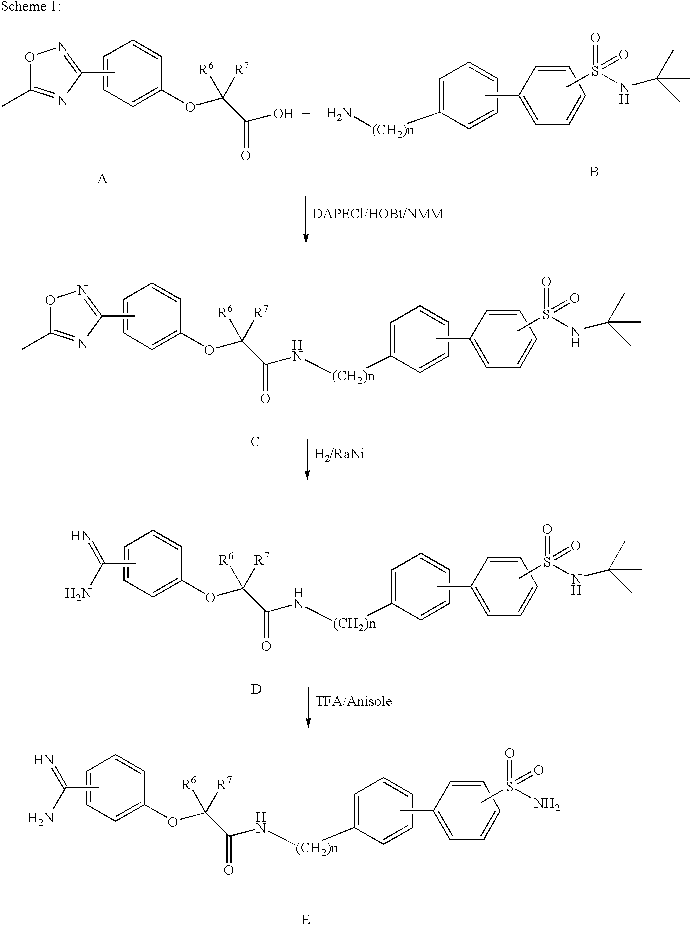

An exemplary synthesis is shown in Scheme 1.

The protected acid unit A is reacted with the amine B with formation of a central amide bond to give the compound C. The carbamimidoyl group is then liberated by reduction with obtainment of the compound D and then the tert-butyl protective group in the acid is removed using trifluoroacetic acid, the active compound E being obtained as the trifluoroacetate.

The acid unit A and the amine B can likewise be prepared according to customary synthesis processes. An exemplary synthesis is presented in Scheme 2 below.

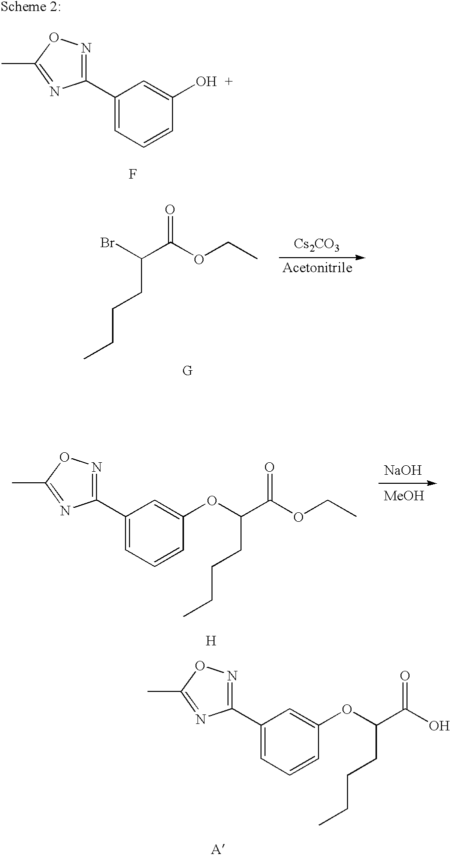

For the synthesis of the acid unit, the phenol derivative F protected on the carbamimidoyl group is reacted with the protected α-bromocarboxylic acid G to give the compound H. The ester H is then hydrolyzed to the carboxylic acid A′.

The amines B can be prepared, for example, in the following way (Scheme 3).

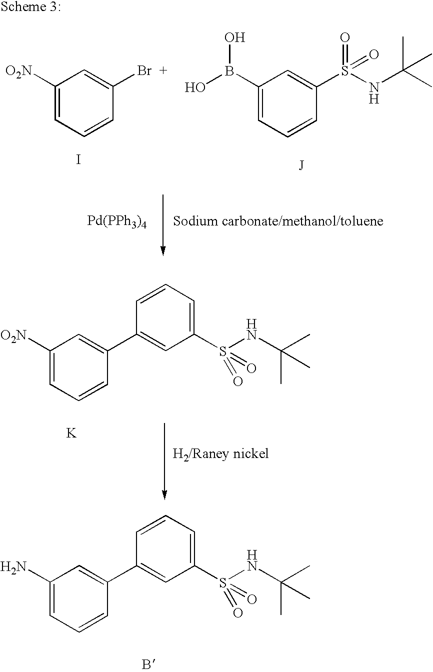

Bromonitrobenzene I is reacted with the boronic acid derivative J to give the biphenyl derivative K. In a further step, the nitro group is reduced to the amine with obtainment of the amine unit B′.

Another suitable synthesis route is shown below (Scheme 4):

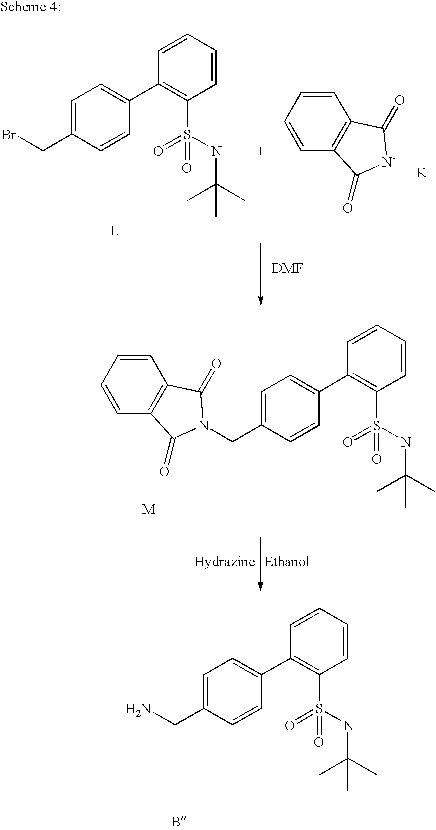

The bromo compound L is reacted with potassium phthalimide to give the compound M. The amine B″ is then liberated from this using hydrazine.

The synthesis routes shown can easily be varied by the person skilled in the art, for example by suitably changing the substitution pattern of the individual synthesis units.

The invention is illustrated in greater detail by means of examples.

### EXAMPLE 1

### 3-[3-N-Hydroxycarbaminidoyl)Phenyl]Propionic Acid

A solution of 60.0 g (342 mmol) of 3-(3-cyanophenyl)propionic acid and 96.0 g (1.38 mol) of hydroxylammonium chloride in 800 ml of ethanol is treated with 180 ml of triethylamine and heated to boiling for 5 hours. The solvent is then removed by distillation and the residue is taken up in water. The precipitated crystals are filtered off and dried in vacuo: 3-[3-(N-hydroxycarbaminidoyl)phenyl]propionic acid as colourless crystals.

### EXAMPLE 2

### 3-[3-(5-Methyl-[1,2,4]oxadiazol-3-yl)phenyl]Propionic Acid

A solution of 30.0 g of (3-[3-(N-hydroxycarbaminidoyl)phenyl]propionic acid in 300 ml of acetic anhydride is heated to boiling for 5 hours. The reaction mixture is concentrated, taken up in water and the precipitated crystals are filtered off with suction: 3-[3-(5-methyl-[1,2,4]oxadiazol-3-yl)phenyl]-propionic acid as colourless crystals, ELMS 232.

### EXAMPLE 3

### 3-[3-(5-Methyl-[1,2,4]oxadiazol-3-yl)phenyl]propionic acid-2′-tert-butylsulfamoylbiphenyl-4-yl)amide

A solution of 200 mg (0.861 mmol) of 3-[3-(5-methyl-[1,2,4]oxadiazol-3-yl)phenyl]propionic acid, 262 mg (0.861 mmol) of 2′-tert-butylsulfamoylbiphenyl-4-yl)amide, 173 mg (0.900 mmol) of N-(3-dimethylaminopropyl)-N′-ethylcarbodiimide hydrochloride (DAPECI) and 122 mg (0.900 mmol) of 1-hydroxybenzotriazole (HOBt) in 2 ml of DMF is treated with 91.0 mg (0.900 mmol) of 4-methylmorpholine and stirred at room temperature for 18 hours. The reaction mixture is added to water and the precipitate is filtered off: 3-[3-(5-Methyl-[1,2,4]oxadiazol-3-yl) phenyl]propionic acid-2′-tert-butylsu lfamoylbiphenyl-4-yl)amide as a colourless solid, FAB 519.

### EXAMPLE 4

### 3-(3-Carbamimidoylphenyl)Propionic Acid-(2′-tert-butylsulfamoylbiphenyl4-yl)Amide Acetate

A solution of 200 mg (0.386 mmol) of 3-[3-(5-methyl-[1,2,4]oxadiazol-3-yl)phenyljpropionic acid-(2′-tert-butylsulfamoylbiphenyl-4-yl)amide in 10 ml of methanol is treated with 100 mg of water-moist Raney nickel and 30 mg of acetic acid and hydrogenated at room temperature and normal pressure for 18 hours. The reaction mixture is filtered and the residue is evaporated. 3-(3-Carbamimidoylphenyl) propionic acid-(2′-tert-butylsulfamoylbiphenyl-4-yl)amide acetate as a colourless solid, FAB 479.

### EXAMPLE 5

### 3-(3-Carbamimidoylphenyl)Propionic Acid-(2+sulfamoylbiphenyl-4-yl)Amide Trifluoroacetate

A solution of 50 mg (0.104 mmol) of 3-[3-(5-methyl-[1,2,4]oxadiazol-3-yl)phenyl]propionic acid-(2′-sulfamoylbiphenyl-4-yl)amide acetate in 1 ml of trifluoroacetic acid is treated with 0.3 ml of anisole and the mixture is stirred at room temperature for 18 hours. The reaction mixture is evaporated, and the residue is stirred with diethyl ether and filtered: 3-(3-carbamimidoylphenyl)propionic acid-(2′sulfamoylbiphenyl-4-yl)amide trifluoroacetate as a colourless solid, FAB 423.

The following examples relate to pharmaceutical preparations.

### EXAMPLE A

### Injection Vials

A solution of 100 g of an active compound of the formula 1 and 5 g of disodium hydrogenphosphate is adjusted to pH 6.5 in 3 l of double-distilled water using 2 N hydrochloric acid, sterile filtered, dispensed into injection vials, lyophilized under sterile conditions and aseptically sealed. Each injection vial contains 5 mg of active compound.

### EXAMPLE B

### Suppositories

A mixture of 20 g of an active compound of the formula I is fused with 100 g of soya lecithin and 1400 g of cocoa butter, poured into moulds and allowed to cool. Each suppository contains 20 mg of active compound.

### EXAMPLE C

### Solution

A solution is prepared from 1 g of an active compound of the formula 1, 9.38 g of NaH₂PO₄₀.2H₂O, 28.48 g of Na₂HPO₄₀.12H₂O and 0.1 g of benzalkonium chloride in 940 ml of double-distilled water. The solution is adjusted to pH 6.8, made up to 1 l and sterilized by irradiation. This solution can be used in the form of eye drops.

### EXAMPLE D

### Ointment

500 mg of an active compound of the formula I are mixed with 99.5 g of petroleum jelly under aseptic conditions.

### EXAMPLE E

### Tablets

A mixture of 1 kg of active compound of the formula I, 4 kg of lactose, 1.2 kg of potato starch, 0.2 kg of talc and 0.1 kg of magnesium stearate is compressed in a customary manner to give tablets such that each tablet contains 10 mg of active compound.

### EXAMPLE F

### Coated Tablets

Tablets are pressed analogously to Example E and are then coated in a customary manner with a coating of sucrose, potato starch, talc, tragacanth and colorant.

### EXAMPLE G

### Capsules

2 kg of active compound of the formula I are filled into hard gelatin capsules in a customary manner such that each capsule contains 20 mg of the active compound.

### EXAMPLE H

### Ampoules

A solution of 1 kg of active compound of the formula I in 60 l of double-distilled water is sterile filtered, dispensed into ampoules, lyophilized under sterile conditions and aseptically sealed. Each ampoule contains 10 mg of active compound.

1. Compounds pf the formula I 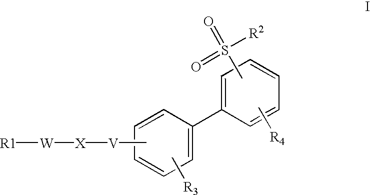
in which:
R¹ is phenyl or naphthyl, which is substituted by —C(═NH)NH₂ (which can also be monosubstituted by —COA, —CO—[C(R⁶)₂—Ar′, —COOA, —OH or by a conventional amino protective group), —NHC(═NH)—NH₂, 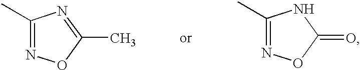
and which can optionally be substituted by —A, —OR⁵, —N(R⁵)₂, —NO₂, —CN, —Hal, —NR⁵COA, —NR⁵COAr′, —NR⁵SO₂A, —NR⁵SO₂Ar′, —COOR⁵, —CON(R⁵)₂, —CONR⁵Ar′, —COR⁶, —COAr′ or —S(O)ₙA;
R² is —N(R⁵)₂, —NR⁵COA, —NR⁵COAr, —NR⁵COOR⁵;
R³,R⁴ independently of one another are —H, —A, —OR⁵, —N(R⁵)₂, —NO₂, —CN, —Hal, —NR⁵COA, —NR⁵COAr′, —NR⁵SO₂A, —NR⁵SO₂Ar′, —COOR⁵, —CON(R⁵)₂, —CONR⁵Ar′, —COR⁶, —COAr′, —S(O)Ar′, —S(O)ₙA;
R⁵ is —H, —A, —C(R⁶R⁷)Ar′ or —C(R⁶R⁷) Het;
R⁶,R⁷ independently of one another are —H, —A or —(CH₂)₁—Ar′;
R⁸ is H or A;
X is —O—, —NR⁵—, —CONR⁵—, —N(SO₂Ar)—, —N(SO₂Het)—;
W is —(CR⁶R⁷)ₙ, —(OCR⁶R⁷)ₒ—, 1,3-phenylene, 1,3-phenylene-C(R⁶)₂—, 1,4-phenylene, 1,4-phenylene-C(R⁶)₂—;
V is —(C(R⁶)₂)ₘ—;
A is alkyl having 1 to 20 C atoms, in which one or two CH₂ groups can be replaced by O or S atoms or by —CH═CH— groups and also 1 to 7H atoms can be replaced by F;
Ar is phenyl or naphthyl, which is unsubstituted or mono-, di- or trisubstituted by —A, —Ar′, —Het, —OR⁵, —N(R⁵)₂, —NO₂, —CN, —Hal, —NR⁵COA, —NR⁵COAr, —NR⁵SO₂A, —NR⁵SO₂Ar′, —COOR⁵, —CON(R⁵)₂, —CONR⁵Ar′, —COR⁶, —COAr′ or —S(O)ₙA;
Ar′ is phenyl or naphthyl, which is unsubstituted or mono-, di- or trisubstituted by —A, —OR⁶, —N(R⁶)₂, —NO₂, —CN, —Hal, —NR⁶COA, —NR⁶SO₂A, —COOR⁶, —CON(R⁶)₂, —COR⁶, —SO₂NR⁶ or —S(O)ₙA;
Het is a mono- or binuclear saturated, unsaturated or aromatic heterocycle having 1 to 4 N, O and/or S atoms, bonded via N or C, which can be unsubstituted or mono-, di- or trisubstituted by —A, —OR⁶, —N(R⁶)₂, —NO₂, —CN, —Hal, —NR⁶COA, —NR⁶SO₂A, —COOR⁶, —CON(R⁶)₂, —COR⁶, —SO₂NR⁶, —S(O)ₙA and/or carbonyl oxygen;
Hal is —F, —Cl, —Br or —l;
l is 0, 1, 2, 3, 4, or 5;
m is 0 or 1;
n is 0, 1 or 2;
o is 1 or 2;
and their pharmaceutically tolerable salts and solvates.

2. Compounds according to claim 1 having the formula II 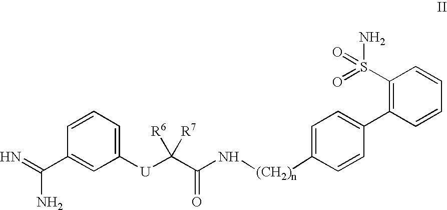
in which in addition:
U is —O— or —CH₂—.

3. Compounds according to claim 1 or 2
2-(3-Carbamimidoylphenoxy)acetic acid (2′-sulfamoylbiphenyl-4-yl)amide (1),
2-(3-Carbamimidoylphenoxy)-2-phenyl-N-(2′-sulfamoylbiphenyl-4-yl)acetamide (2),
2-(3-Carbamimidoylphenoxy)valeric acid (2′-sulfamoylbiphenyl-4-yl)amide (3),
2-(3-Carbamimidoylphenoxy)hexanoic acid (2′-sulfamoylbiphenyl-4-yl)amide (4),
2-(3-Carbamimidoylphenoxy)heptanoic acid (2′-sulfamoylbiphenyl-4-yl)amide (5),
2-(3-Carbamimidoylphenoxy)-3-methyl-N-(2′-sulfamoylbiphenyl-4-yl)butyramide (6),
2-(3-Carbamimidoylphenoxy)-2-methylvaleric acid (2′-sulfamoyl-biphenyl-4-yl)amide (7),
2-(3-Carbamimidoylphenoxy)-2-phenyl-N-(2′-sulfamoylbiphenyl-4-yl)acetamide (8),
2-(3-Carbamimidoylphenoxy)-4-phenyl-N-(2′-sulfamoylbiphenyl-4-yl)butyramide (9),
2-(3-Carbamimidoylphenoxy)-2-methyl-N-(2′-sulfamoylbiphenyl-4-yl)propionamide (10),
3-(3-Carbamimidoylphenoxy)propionic acid (2′-sulfamoylbiphenyl-4-yl)amide (11),
2-(3-Carbamimidoylbenzyl)pentanoic acid (2′-sulfamoylbiphenyl-4-yl)amide (12×),
3-(3-Carbamimidoylphenyl)-2-phenyl-N-(2′-sulfamoylbiphenyl-4-yl)propionamide (13),
2-Benzyl-3-(3-carbamimidoylphenyl)-N-(2′-sulfamoylbiphenyl-4-yl)propionamide (14),
2-(3-Carbamimidoylbenzyl)-N-(2′-sulfamoylbiphenyl-4-yl)butyramide (65),
2-(3-Carbamimidoylbenzyl)-4-methylpenanoic acid (2′-sulfamoylbiphenyl-4-yl)amide (66),
2-(3-Carbamimidoylphenoxy)acetic acid (2′-sulfamoylbiphenyl-4-ylmethyl)amide (15),
2-(3-Carbamimidoylphenoxy)-N-(2′-sulfamoylbiphenyl-4-ylmethyl)propionamide (16),
2-(3-Carbamimidoylphenoxy)-N-(2′-sulfamoylbiphenyl-4-ylmethyl)butyramide (17),
2-(3-Carbamimidoylphenoxy)pentanoic acid (2′-sulfamoylbiphenyl-4-ylmethyl)amide (18),
2-(3-Carbamimidoylphenoxy)-3-methyl-N-(2′-sulfamoylbiphenyl-4-ylmethyl)butyramide (19),
2-(3-Carbamimidoylphenoxy)-4-methylpentanoic acid (2′-sulfamoyl-biphenyl-4-ylmethyl)amide (20),
2-(3-Carbamimidoylphenoxy)-2-phenyl-N-(2′-sulfamoylbiphenyl-4-ylmethyl)acetamide (21),
2-(3-Carbamimidoylphenoxy)propionic acid (3′-sulfamoylbiphenyl-4-yl)amide (22),
2-(3-Carbamimidoylphenoxy) butyric acid (3′-sulfamoylbiphenyl-4-yl)amide (23),
2-(3-Carbamimidoylphenoxy)valeric acid (3′-sulfamoylbiphenyl-4-yl)amide (24),
2-(3-Carbamimidoylphenoxy)-4-methylvaleric acid (3′-sulfamoyl-biphenyl-4-yl)amide (25),
2-(3-Carbamimidoylphenoxy)-2-phenylacetic acid (3′-sulfamoyl-biphenyl-4-yl)amide (26),
2-(3-Carbamimidoylphenoxy)-N-(3′-sulfamoylbiphenyl-3-yl)butyramide (27),
2-(3-Carbamimidoylphenoxy)pentanoic acid (3′-sulfamoylbiphenyl-3-yl)amide (28),
2-(3-Carbamimidoylphenoxy)-4-methylpentanoic acid (3′-sulfamoyl-biphenyl-3-yl)amide (29),
2-(3-Carbamimidoylphenoxy)-2-phenyl-N-(3′-sulfamoylbiphenyl-3-yl)acetamide (30),
2-(4-Carbamimidoylphenoxy)pentanoic acid (2′-sulfamoylbiphenyl-4-yl)amide (31),
2-(4-Carbamimidoylphenoxy)-2-phenyl-N-(2′-sulfamoylbiphenyl-4-yl)acetamide (32),
3-Carbamimidoylbenzoic acid (2′-sulfamoylbiphenyl-4-yl)amide (33),
2-(3-Carbamimidoylphenyl)acetic acid (2′-sulfamoylbiphenyl-4-yl)amide (34),
4-Carbamimidoylbenzoic acid (2′-sulfamoylbiphenyl-4-yl)amide (35),
2-(4-Carbamimidoylphenyl)acetic acid (2′-sulfamoylbiphenyl-4-yl)amide (36),
3-(4-Carbamimidoylphenyl)propionic acid (2′-sulfamoylbiphenyl-4-yl)amide (37),
2-(4-Carbamimidoylphenoxy)acetic acid (2′-sulfamoylbiphenyl-4-yl)amide (38),
3-(3-Carbamimidoylphenyl)propionic acid (2′-sulfamoylbiphenyl-4-ylmethyl)amide (39),
2-(3-Carbamimidoylphenyl)acetic acid (2′-sulfamoylbiphenyl-4-ylmethyl)amide (40),
2-(4-Carbamimidoylphenyl)acetic acid (3′-sulfamoylbiphenyl-4-yl)amide (41),
2-(3-Carbamimidoylphenyl)acetic acid (3′-sulfamoylbiphenyl-4-yl)amide (42),
3-(3-Carbamimidoylphenyl)propionic acid (3′-sulfamoylbiphenyl-4-yl)amide (43),
2-(3-Carbamimidoylphenoxy)acetic acid (3′-sulfamoylbiphenyl-4-yl)amide (44),
4-(2′-Sulfamoylbiphenyl-3-yloxymethyl)benzamidine (45),
3-(2′-Sulfamoylbiphenyl-3-yloxymethyl)benzamidine (46),
4-(2′-Sulfamoylbiphenyl-4-ylmethoxy)benzamidine (47),
3-(2′-Sulfamoylbiphenyl-4-ylmethoxy)benzamidine (48),
3-(3-Carbamimidoylphenoxy)-N-(2′-sulfamoylbiphenyl-4-yl)propionamide (67),
2-(4-Carbamimidoylphenyl)acetic acid (2′-sulfamoylbiphenyl-3-yl)amide (49),
2-(3-Carbamimidoylphenyl)acetic acid (2′-sulfamoylbiphenyl-3-yl)amide (50),
3-(4-Carbamimidoylphenyl)propionic acid (2′-sulfamoylbiphenyl-3-yl)amide (51),
3-(3-Carbamimidoylphenyl)propionic acid (2′-sulfamoylbiphenyl-3-yl)amide (52),
2-(4-Carbamimidoylphenoxy)acetic acid (2′-sulfamoylbiphenyl-3-yl)amide (53),
2-(3-Carbamimidoylphenoxy)acetic acid (2′-sulfamoylbiphenyl-3-yl)amide (54),
7-(2′-Sulfamoylbiphenyl-4-yloxymethyl)naphthalene-2-carboxamidine (55),
7-(2′-Sulfamoylbiphenyl-4-yloxymethoxy)naphthalene-2-carboxamidine (56),
7-(2′-Sulfamoylbiphenyl-4-ylaminomethyl)naphthalene-2-carboxamidine (57),
7-(2′-Sulfamoylbiphenyl-3-yloxymethyl)naphthalene-2-carboxamidine (58),
3′-(2′-Sulfamoylbiphenyl-4-ylaminomethyl) biphenyi-3-carboxamidine (59),
3′-(2′-Sulfamoylbiphenyl-4-yloxymethyl)biphenyl-3-carboxamidine (60),
N-(4-Ethylbenzenesulfonyl)-3′-(2′-sulfamoylbiphenyl-4-ylaminomethyl)biphenyl-3-carboxamidine (61),
3′-(2′-Sulfamoylbiphenyl-3-yloxymethyl)biphenyl-3-carboxamidine (62),
3′-Carbamimidoylbiphenyl-3-carboxylic acid (2′-sulfamoylbiphenyl-3-yl)amide (63),
3′-Carbamimidoylbiphenyl-3-carboxylic acid (2′-sulfamoylbiphenyl-4-yl)amide (64),
2-(3-Carbamimidoyl-benzyl)-N-(2′-sulfamoyl-biphenyl-4-yl)butyramide (65),
2-(3-Carbamimidoyl-benzyl)-4-methylpentanoic acid(2′-sulfamoyl-biphenyl-4-yl)amide (66),
3-(3-Carbamimidoyl-phenoxy)-N-(2′-sulfamoyl-biphenyl-4-yl)propionamide (67),
2-(3-Carbamimidoylbenzyl)hexanoic acid (2′-su lfamoylbiphenyl-4-yl)amide (68),
3-{1-[(2′-Sulfamoylbiphenyl-4-ylamino)methyl]butoxy}benzamidine (69).

4. Compound according to one of claims 1 to 3 as a pharmaceutical active compound.

5. Use of a compound according to one of claims 1 to 3 for the production of a medicament for the treatment of thromboses, myocardial infarct, arteriosclerosis, inflammation, apoplexy, angina pectoris, resteriosis after angioplasty and intermittent claudication.

6. Process for the production of pharmaceutical preparations, in which a compound according to one of claims 1 to 3 and/or one of its physiologically acceptable salts is converted into a suitable dose form together with at least one solid, liquid or semi-liquid vehicle or excipient.

7. Compound according to one of claims 1 to 3 as an inhibitor of coagulation factor Xa.

8. Compound according to one of claims 1 to 3 as an inhibitor of coagulation factor VIIa.

9. Pharmaceutical preparation comprising at least one compound according to one of claims 1 to 3 or one of its physiologically acceptable salts.
